(19) United States
(12) Patent Application Publication
(10) Pub. No.: US 2024/0278016 A1
(43) Pub. Date: Aug. 22, 2024

(54) DEEP BRAIN STIMULATION (DBS) METHOD AND DEVICE

(71) Applicant: Worcester Polytechnic Institute, Worcester, MA (US)

(72) Inventors: Sergey N. Makaroff, Holden, MA (US); Manas Rachh, New York, NY (US); Leslie Greengard, New York, NY (US); Laleh Golestani Rad, Wilmette, IL (US)

(21) Appl. No.: 18/581,910

(22) Filed: Feb. 20, 2024

Related U.S. Application Data
(60) Provisional application No. 63/447,018, filed on Feb. 20, 2023.

(51) Int. Cl. A61N 1/36; A61N 1/05; G16H 20/40

(52) U.S. Cl. CPC A61N 1/36128; A61N 1/0534; A61N 1/36067; G16H 20/40

(57) ABSTRACT

A method for directing an electrostimulation therapy includes receiving a scan image of a treatment region and determining a purported location of a stimulation probe inserted within the scan image. A modeling application or computational engine determines a position of a target region within the scan image relative to the purported location, and computes a strength of the electrical energy at the position based on a Fast Multipole Method (FMM) using LU (Lower/Upper) factorization, and concludes an efficacy resulting from activation of an electrode delivering the electrical energy resulting from the stimulation probe at the purported location.

### RELATED APPLICATIONS

This patent application claims the benefit under 35 U.S.C. § 119(e) of U.S. Provisional Patent App. No. 63/447,018 filed Feb. 20, 2023, entitled “DEEP BRAIN STIMULATION SIMULATOR” incorporated herein by reference in entirety.

### BACKGROUND

Deep Brain Stimulation involves implanting electrodes within areas of the brain for therapeutic effects. The electrodes produce electrical impulses that affect brain activity to treat certain medical conditions. The electrodes emit an electric field that affect neurons, particularly the axon, in a way that can be beneficial for treatment of certain disorders. The emitted electric field and corresponding effects on proximal neurons is complex.

Accordingly, multiscale modeling of DBS is an active area of research. DBS has shown particular promise in the treatment of Parkinson's disease, for example. The motor hyper-direct pathway (HDP), which directly connects the motor cortex to the subthalamic nucleus (STN), is considered a key target in the treatment of Parkinson's disease symptoms with DBS. Recently, biophysical models of the human HDP have been used to explore the therapeutic mechanisms of subthalamic DBS. However, comparison of clinical and model-predicted thresholds for evoked potentials implies that model detail would benefit from more precise prediction of pathway recruitment.

### SUMMARY

A DBS modeling approach provides real-time (RT) or near RT modeling results for an electrode probe inserted into a neural treatment region for determining the resulting electrical field induced efficacy at various neurons (axons) in the treatment region around the electrodes. Neurons follow a varied, non-linear path through brain tissue. Based on a scan of the neurons, which are typically a bundled structure, the disclosed approach computes and models an electric field emanating from a plurality of electrodes on a probe inserted through or near the bundle. In treatment of Parkinson's disease (Parkinson's), treatment targets the HDP, and modeling includes an electric field emanating from 4 or 8 electrode bands on an inserted probe emanating a pulsed electric signal. A Fast Multipole Method (FMM) with LU (lower/upper) factorization defines an FMM-LU approach for determining the electric fields generated by different voltage combinations substantially faster than the prior iterative BEM-FMM (Boundary Element Method-FMM), and particularly faster than conventional Finite Element Methods (FEM) approaches.

Configurations herein are based, in part, on the observation that DBS has shown particular promise in the treatment of neurological disorders such as Parkinson's disease (Parkinson's) and depression. DBS includes determining an electrical field over a treatment region into which the electrodes are inserted. Unfortunately, conventional approaches to modeling DBS electric fields suffer from the shortcoming of a non-deterministic, trial-and-error approach, requiring up to 6 months of iterative testing. The problem is exacerbated by the emergence of a new generation of DBS devices exponentially increasing the number of possible combinations, making it computationally infeasible to find the optimum setting by trial and error within a timely manner.

Accordingly, configurations herein substantially overcome the shortcomings of conventional DBS modeling approaches by providing an FMM-based LU factorization, or FMM-LU approach, which improves the time to model the generated DBS fields by around two orders of magnitude. The use of FMM-LU is depicted for an example treatment of Parkinson's, however other electric-field based treatments of neural or other anatomical structures may benefit from the techniques disclosed herein. In the disclosed examples, the HDP is a key target in the treatment of Parkinson's using (DBS). Biophysical models of HDP DBS have been used to explore the mechanisms of stimulation.

In a particular configuration, a method for directing an electrostimulation therapy includes receiving a scan image of a treatment region and determining a purported location of a stimulation probe inserted within the scan image. A modeling application or computational engine determines a position of a target region within the scan image relative to the purported location, and computes a strength of the electrical energy at the position based on the FMM-LU approach, and concludes an efficacy resulting from activation of an electrode delivering the electrical energy resulting from the stimulation probe at the purported location.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a DBS treatment environment for amenable neurological treatment;

FIG. 2 shows an example of electrode placement for DBS treatment environment for Parkinson's, showing electrode types operable for modeling, including: (i) imprinted TES surface electrodes, (ii) embedded ICMS electrode arrays of (planar or laminar), and (iii) embedded DBS electrodes;

FIGS. 3A-3B show the electric field emanated from the stimulation probe of FIG. 2;

FIGS. 4A-4C show the stimulation probe of FIGS. 3A-3B and the brain structures of FIG. 2 in greater detail;

FIGS. 5A-5B show a shaded graph of electric field strength at an axon in the neural structure of FIG. 4A;

FIG. 6 shows modeling of the brain and inserted electrode resulting from the scan image of FIGS. 4A-4C; and

FIG. 7 shows variances in neural (brain) tissue modeled by the disclosed approach.

### DETAILED DESCRIPTION

Conventional approaches to DBS modeling for treatment of Parkinsons's employ a finite element method (FEM) high-resolution modeling technique for electrical brain stimulation. Improved approaches to DBS modeling include the boundary element fast multipole method (BEM-FMM) and are further enhanced using LU (Lower/Upper) factorization in an FMM-LU approach, disclosed further below. The result is a practical electrode model for both surface and embedded electrodes.

Integral equations for computing a surface charge density are combined with a general-purpose fast multipole method and LU factorization and may be expanded for voltage, shunt, current, and floating electrodes. The resulting solution of coupled and properly weighted/preconditioned integral equations is accompanied by enforcing global conservation laws: charge conservation law and Kirchhoff's current law.

Brain stimulation therapies are important and effective treatments for people with Parkinson's, depression and other mental disorders. Along with critical applications related to the senior population, depression has been the leading cause of disability in the US among young people. Patients with depression who have failed to receive benefit from medications have experienced a clinically meaningful response with brain stimulation. Over the past fifteen years, the number of brain stimulation devices to undergo the FDA approval processes has grown exponentially in number and has shown significant sustained interest. In particular, improvements have been noted for the most challenging implanted invasive devices: those targeting Parkinsonian symptoms and tremors. Other demanding clinical applications include presurgical mapping in epileptic patients and accurate motor mapping prior to brain tumor surgery, as well as brain-computer interfaces.

Brain stimulation therapies include Transcranial electrical stimulation (TES)—including transcranial direct current stimulation (tDCS) and transcranial alternating current stimulation (tACS)—a low-cost portable application technique with applied currents usually less than 1-2 mA. Another approach involves Cortical Stimulation (CS) and intracortical microstimulation (ICMS)-invasive yet precise versions of TES with smaller injected currents. Small implanted electrodes may target/activate selected populations or nuclei of neurons and have applications in brain and motor mapping pertinent to epilepsy.

However, one of the most challenging approaches involves Deep Brain Stimulation (DBS)—an invasive technique with a permanently implanted neurostimulator targeting deep parts of the brain such as the subthalamic nucleus and forebrain bundle to reduce symptoms of treatment resistant depression and Parkinson's disease. The success of subthalamic deep brain stimulation for Parkinson's disease is highly dependent on knowledge of the anatomical extent of the electric field surrounding the active electrode contact with brain tissue. Since this involves directly implanted electrodes, it is important to model the electrical field, and particularly surface charge density, on a neural axon in the field induced by the implanted electrode.

DBS simulators work by precalculating the distribution of electric fields in the tissue on a standard model, saving those values, and estimating the response of a new patient based on the pre-calculated data. The reason for this is that solving a typical FEM problem involving a DBS lead model in a simple tissue environment takes a few hours, and thus, cannot be applied in real time in patients. The BEM-based FMM-LU approach herein, on the other hand, takes less than a minute to solve the same problem. This means that the FMM-LU solver can be applied in real-time in patients, taking into account the specific patient data rather than relying on pre-calculated, often inaccurate data from a standard patient model.

In conventional approaches, oversimplified axonal anatomy and branching might explain much of the prediction error. Heterogeneous charge deposition and voltage-gated channel distribution on variegated membrane surfaces, such as at bifurcations and terminals, may partly explain lingering errors in predictions of axon recruitment in response to extracellular electric fields. Built upon finite element method (FEM) volume conductor solutions, models of DBS pathway recruitment are often limited by a resolution mismatch which ignores local charge deposition on neuronal membranes. Further, FEM models, which can accurately estimate charge deposition on anatomically realistic axons at the micron-scale, are computationally expensive. Lastly, the spatial derivative of the external macroscopic electric field is frequently used as an estimator of neuronal recruitment (activating function), ignoring the effect of unique neuronal geometry on membrane polarization.

Configurations herein demonstrate that the FMM-LU approach can be employed in modeling of neurophysiological recordings and neurostimulation. To this end, the FMM-LU method is formulated in terms of surface charge densities at the conductivity interfaces. At present, it is possible to solve systems with about 100 million facets in several hours. Extensions to anisotropic media are possible via the method of volume integral equation. The FMM-LU method may outperform the commonly used finite element modeling method for three types of problems. The first (mesoscale) type implies a large number of piecewise homogeneous tightly-spaced mesoscale compartments such as, for example, thin brain meninges and other extracerebral brain compartments. The second (multiscale) type implies microscale objects such as an intracortical microarray embedded into a macroscopic model. The third (microscale) type is pertinent to modeling large ensembles of realistic axonal/dendritic arbor of a very complicated geometry.

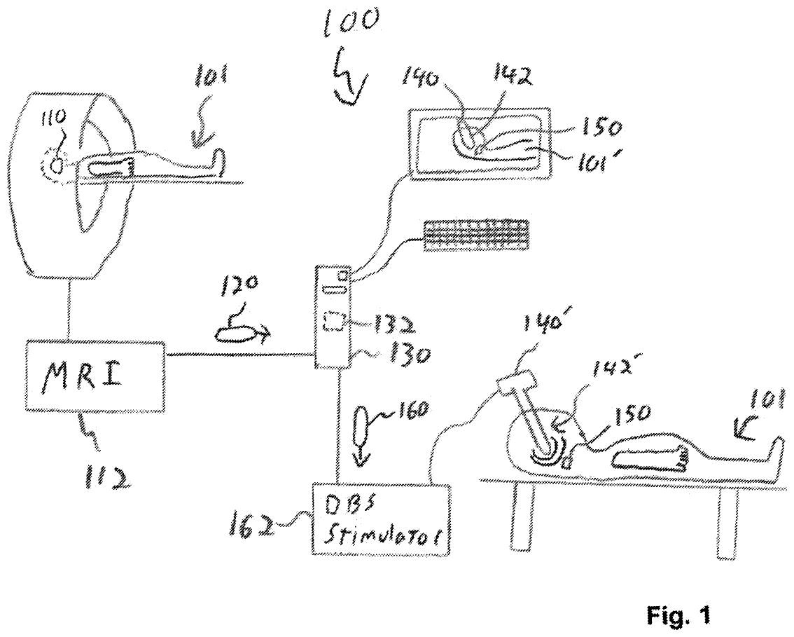

FIG. 1 is a context diagram of a DBS treatment environment 100 for amenable neurological treatment. Referring to FIG. 1, the disclosed method for directing an electrostimulation therapy such as DBS includes receiving a scan image 120 of a treatment region 110 of a patient 101. Any suitable scanning or imaging medium may be employed, such as from an MRI (Magnetic Resonance Imaging) device 112, a CT/CAT (Computerized Tomography/Computerized Axial Tomography), Ultrasound, or other approach for generating a scan image 120 in a pixelated 3-dimensional form.

The scan image 120 is received by a modeling application 132 launched on a server 130. From the scan image 120, the modeling application 132 computes or determines a purported location 142 of a stimulation probe 140 having electrodes inserted within the scan image 120. Typically this involves an insertion depth of the stimulation probe 140 that disposes banded electrodes on the probe (typically 4) at a particular location, Based on the scan image, the modeling application 132 determines a position 150 of a target region within the scan image 120 relative to the purported location of the electrodes as imaged on the patient 101′. The modeling application 132 computes a strength of the electrical energy at the position 150 based on the electric field at the position 150. In contrast to conventional approaches, this may involve computing a strength of the electrical energy at the position based on a derivative of the value representing the electric field at the position. Conventional approaches are burdened by the need to compute the derivative for determining the effect on the axon at the position.

The target region may be defined by a pixel in the scan image 120 and a corresponding relative position from the electrode. In the disclosed approach, the scan image 120 is a pixelated structure including a plurality of pixels and the target region includes a pixel at the position 150. A full treatment regimen would entail iterating over a plurality of positions 150 depicted by the scan image 120, to fully assess the electric field emanating from the electrode based on the modeled location 142 of the stimulation probe 140. This applies to each electrode on the stimulation probe 140 (typically 4), discussed further below.

As the modeling application 132 computes the electric field based on a certain probe location 142 and scan image 120 of a patient, additional modeling may be performed until the actual DBS procedure is performed by inserting a live probe 140′ into a therapeutically optimal position 142′ on the live patient 101. Hence, based on the position of an electrode, the corresponding electric field affecting a brain tissue region at the position 150 depicted on the scan image 120 is computed. Iterative application of the modeling may adjust the purported location 142 of the stimulation probe 140 to an alternate purported location, and re-evaluate the efficacy based on the stimulation probe 140 being disposed in the alternate purported location 142. The modeling application 132 concludes an efficacy resulting from activation of an electrode delivering the electrical energy from the stimulation probe 140 at the purported location 142, before the invasive procedure of actual DBS using a surgically inserted stimulation probe 140 into the patient 101. Concluding the efficacy can further include comparing the computed strength of the electric field to a threshold indicative of a therapeutic effect on the treatment region 110, for example to position the electrodes for maximal effect on a particular axon structure.

Once the modeling of the electric field has computed an effective position and voltage profile for DBS delivery, a set of DBS instructions 160 is issued to a DBS stimulator 162 for driving the stimulation probe 140′ inserted to the computed location 142′ by applying therapeutic voltage to the electrodes at a voltage level and pulse rate set by the instructions 160.

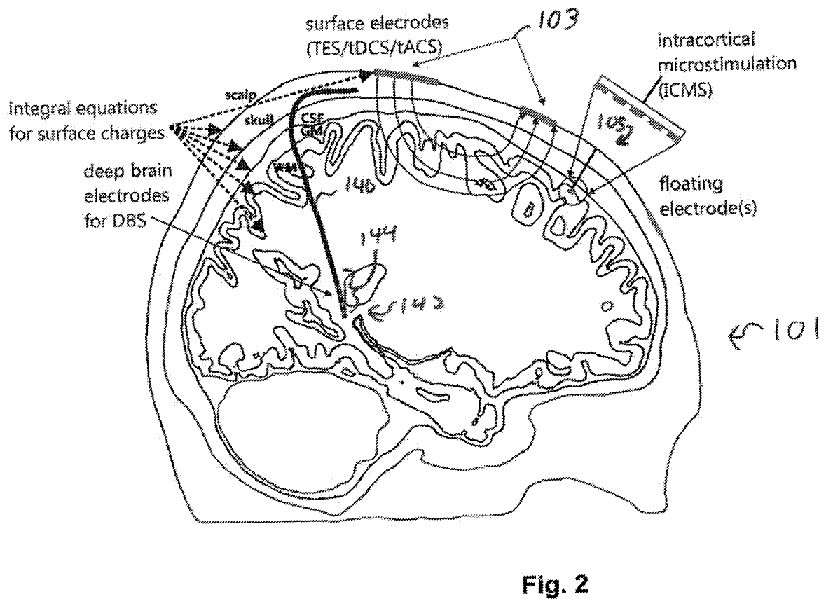

FIG. 2 shows an example of electrode placement for DBS treatment environment for Parkinson's, showing electrode types operable for modeling, including: (i) imprinted TES surface electrodes, (ii) embedded ICMS electrode arrays of (planar or laminar), and (iii) embedded DBS electrodes. Referring to FIGS. 1 and 2, electrode based therapy on the patient 101 may include surface electrodes 103 for TES, and ICMS electrodes 105. The disclosed approach employs a stimulation probe 140 inserted into brain tissue to a location 142 for deploying one or more electrodes 144, discussed further below.

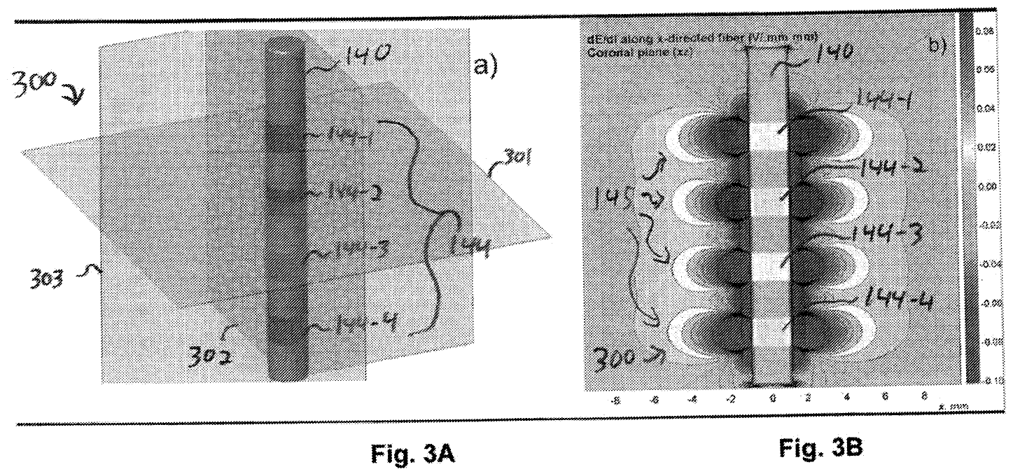

FIGS. 3A-3B show the electric field emanated from the stimulation probe of FIG. 2. Referring to FIGS. 3A-3B, the volume around the probe 140 defines a 3-dimensional volumetric region 300, referenced as dimensions depicted by a horizontal plane 301, a first vertical plane 302, and a second vertical plane 303 orthogonal to the other first vertical plane 302 and the horizontal plane 301. Electric fields emanate from each of 4 electrodes 144-1 . . . 144-4 (144 generally), which are implemented as conductive bands around the stimulation probe 140. FIG. 3B shows a shaded graph of an electromagnetic field 145 emanating from each electrode 144-1 . . . 144-4.

The modeling application 132 invokes the scan image 120 and the volumetric region 300 around the probe 140 at a given location 142 to determine the electric field 145 of a target region (position) relative to the probe 140. It can be seen that the stimulation probe 140 includes a plurality of electrodes 144, and that the modeling application 132 computes the strength of the electrical energy contributed from each of the plurality of electrodes 144. In an example configuration, the plurality of electrodes further includes at least one energized electrode, the energized electrode engaged with a voltage or current source, and at least one floating electrode contributing electrical energy in an absence of electrical communication with the voltage or current source. The floating electrodes are not directly connected to the DBS stimulator 162 or other voltage source, and exhibit a constant and unknown voltage with a zero net current. A particular configuration deploys the floating electrodes 144-2, 144-3 flanked by sourced electrodes 144-1, 144-4 connected to a voltage source.

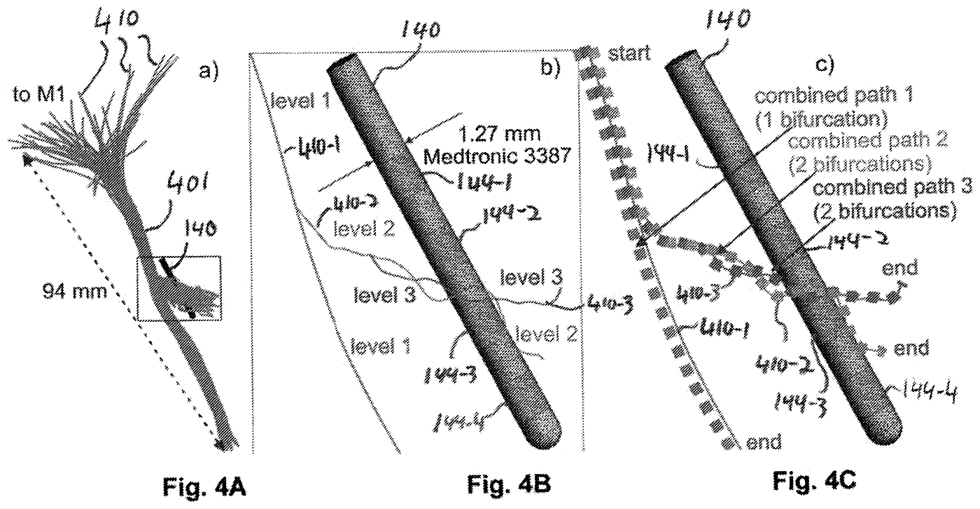

FIGS. 4A-4C show the stimulation probe of FIGS. 3A-3B and the brain structures of FIG. 2 in greater detail. Referring to FIGS. 1-4C, FIG. 4A depicts the HDP 401. The HDP represents the main glutamatergic input to the subthalamic nucleus (STN), through which the motor and prefrontal cerebral cortex can modulate basal ganglia activity. Further, direct activation of the motor HDP is a key target in the treatment of Parkinson's disease with DBS. Biophysical models of HDP DBS have been used to explore the mechanisms of stimulation. The HDP 401 at a basic level is a bundle of neurons 410 where the DBS electric field targets the axon of the neurons. The stimulation probe 140 is preferably inserted near or through this bundle, shown in FIG. 4A. FIGS. 4B and 4C show proximity of individual neurons 410-1 . . . 410-3 following an irregular, non-linear path around the probe 140 in various positions relative to the electrodes 144-1 . . . 144-4.

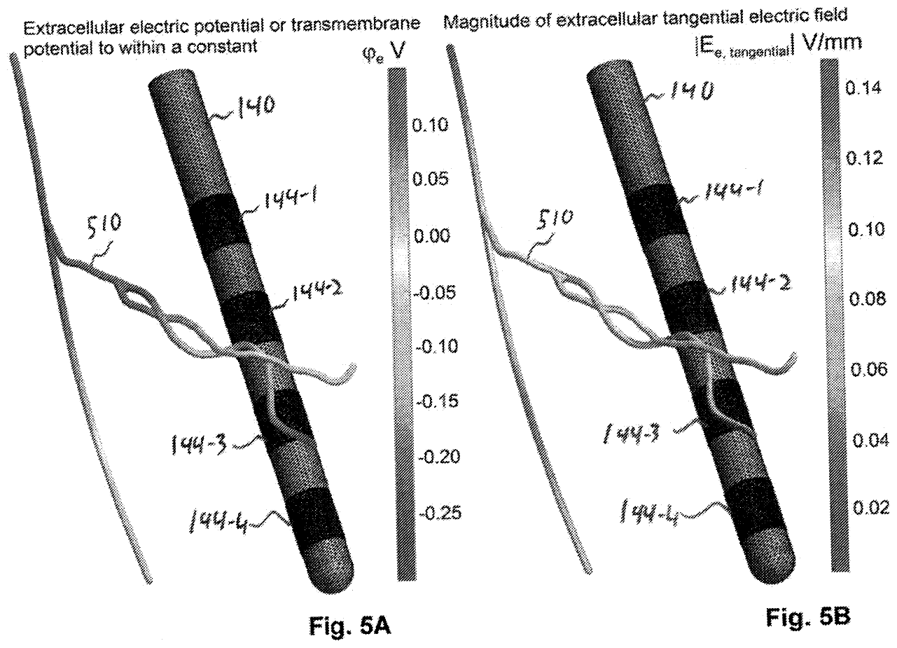

FIGS. 5A-5B show a shaded graph of electric field strength at an axon 510 in the neural structure of FIG. 4A. Referring to FIGS. 4A-5B, the shading shows computations for a 2 μm thick axon projected onto the corresponding tubular surface with a much larger diameter of 200 μm for visualization purposes. Electrode 144-4 (bottom) is a cathode, electrode 144-1 (top) is an anode at 1 mA of the net current. In FIG. 5A, extracellular electric potential at the end of initial polarization is shown for axon 510. The transmembrane potential is this value minus the axon resting potential. The transmembrane potential is simultaneously the double layer dipole density to within a multiplicative constant—the dielectric permittivity. FIG. 5B shows a magnitude of extracellular electric field at the end of initial polarization for the axon 510.

Configurations herein, therefore, employ the fast multipole method based computational engine to model a typical DBS topology (leads, subthalamic nucleus, a part of the motor hyperdirect pathway, thalamus) in approximately 0.2-0.5 sec. The disclosed use of FMM-based LU factorization or FMM-LU allows determination of the fields generated by different voltage combinations 20-30 times faster than the prior iterative BEM-FMM.

The disclosed FMM-LU approach performed by the modeling application 132 includes the following:

Determining Fredholm integral equation of the second kind at all conductivity interfaces S\Sₑ with contrasts K in terms of induced charge density ρ(r):

$$\frac{ρ ( r )}{2} - K ( r ) n ( r ) \cdot {\int }_{S} \frac{1}{4 π} \frac{r - {r}^{′}}{{\vert r - {r}^{′} \vert}^{3}} ρ ( {r}^{′} ) d {r}^{′} = 0 , r ∈ S ∖ {S}_{e}$$

Establishing Fredholm integral equation of the first kind at electrode surfaces Sₑ with voltage V in terms of induced charge density ρ(r)

$$\frac{1}{4 π {ε}_{0}} {\int }_{S} \frac{ρ ( {r}^{′} )}{\vert r - {r}^{′} \vert} d {r}^{′} = V , r ∈ {S}_{e}$$

Next is to discretize integral equations at surface facets with zeroth-order pulse bases in the form Ax=b:

$$\begin{matrix}[ \begin{matrix}{ℝ}_{mn} = \int {\int }_{{A}_{m} {A}_{n}}^{} \frac{1}{4 {πε}_{0}} \frac{( r - {r}^{′} )}{{\vert r - {r}^{′} \vert}^{3}} {dr}^{′} dr \\ \\ m = 1 : M , n = 1 : M + N\end{matrix} ] \\ [ \begin{matrix}{ℤ}_{mn} = \frac{1}{2} {δ}_{mn} - \frac{K}{{A}_{m}} {n}_{m} \cdot \int {\int }_{{A}_{m} {A}_{n}}^{} \frac{1}{4 π} \frac{( r - {r}^{′} )}{{\vert r - {r}^{′} \vert}^{3}} {dr}^{′} dr \\ \\ m = M + 1 : M + N , n = 1 : M + N\end{matrix} ]\end{matrix} \times [ \begin{matrix}{c}_{1} \\ {c}_{2} \\ {c}_{3} \\ … \\ … \\ … \\ … \\ … \\ … \\ … \\ {c}_{M + N}\end{matrix} ] = \begin{matrix}\begin{matrix}[ {\int }_{{A}_{m}}^{} Vdr ] \\ m = 1 : M\end{matrix} \\ [ \begin{matrix}0 \\ 0 \\ 0 \\ … \\ 0\end{matrix} ]\end{matrix}$$

The approach applies FMM to a direct LU-factorization of matrix A.

FMM-based LU factorization, or FMM-LU, finds a compressed approximation of A⁻¹ directly so that a direct solution x=A⁻¹b is attempted, without using an iterative method and for multiple right-hand sides, b.

The resulting model thus computes the DBS voltage instructions 160 for a stimulation probe 140′ placed at a location 142′ without a substantial delay, allowing same-session turnaround for the DBS patient 101.

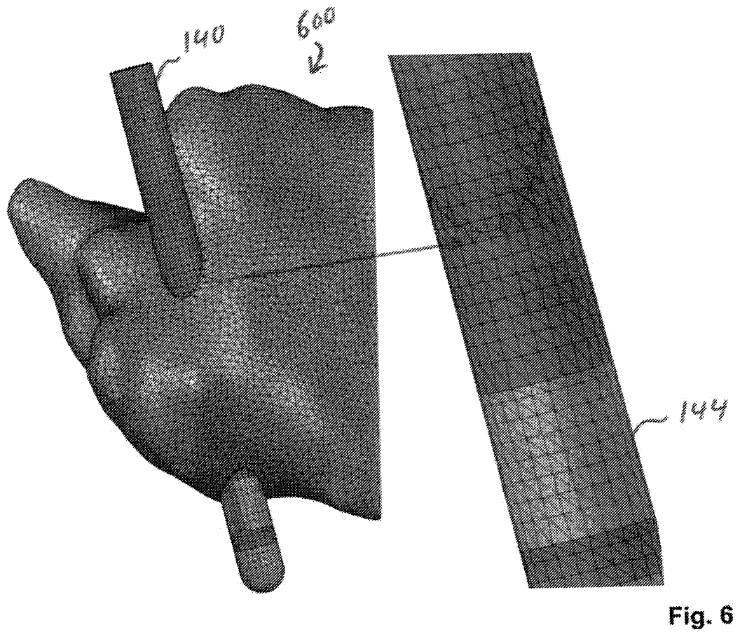

FIG. 6 shows modeling of the brain and inserted electrode resulting from the scan image of FIG. 4. In a particular approach, computation of the strength of the electrical energy is based on a faceted volumetric representation of the treatment region. Referring to FIGS. 6, FIG. 6 shows a faceted region 600 as employed by the modeling application 132 for computing a surface charge density at each of a particular facet in the 3D region 300 within the electric fields 145 of the respective electrodes. Computation of the strength of the electrical energy is based on determining a surface charge density at a position of a neural axon in the treatment region.

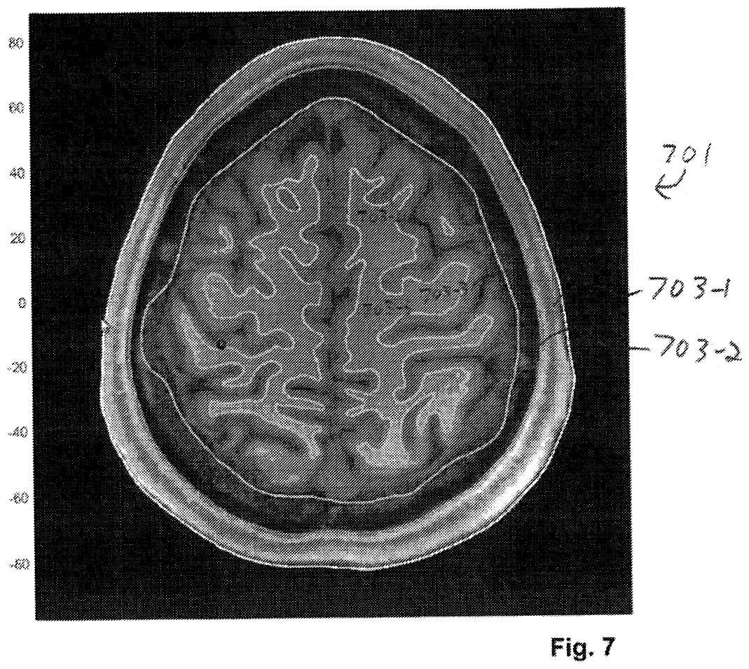

FIG. 7 shows variances in neural (brain) tissue modeled by the disclosed approach. Referring to FIGS. 1 and 7, the scan image 120 depicting the brain of the patient 101 may further define regions of tissue variances, shown in a tissue density graph 701. Conventional approaches consider a homogenous volume or structure to neural/bran tissue, and thus assume equal propagation of an electrical field 145 through the tissue. The scan image 120, in contrast, can depict different tissue densities in regions 703-1 . . . 703-5. Alternate scales and grading of tissue density may be effected; it is noteworthy that the scan can accommodate electrical field propagation and absorption across differing tissue densities and/or types.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for directing an electrostimulation therapy, comprising:
receiving a scan image of a treatment region;
determining a purported location of a stimulation probe inserted within the scan image;
determining a position of a target region within the scan image relative to the purported location;
computing a strength of the electrical energy at the position based on the electric field at the position; and
concluding an efficacy resulting from activation of an electrode delivering the electrical energy resulting from the stimulation probe at the purported location.

2. The method of claim 1 wherein concluding the efficacy further comprises comparing the computed strength to a threshold indicative of a therapeutic effect on the treatment region.

3. The method of claim 1 wherein the scan image is a pixelated structure including a plurality of pixels and the target region includes a pixel at the position.

4. The method of claim 1 further comprising iterating over a plurality of positions depicted by the scan image.

5. The method of claim 4 further comprising:
adjusting the purported location of the stimulation probe to an alternate purported location; and
re-evaluating the efficacy based on the stimulation probe being disposed in the alternate purported location.

6. The method of claim 1 wherein the target region is an axon in a human brain.

7. The method of claim 1 wherein the stimulation probe includes a plurality of electrodes on the stimulation probe, further comprising computing the strength of the electrical energy contributed from each of the plurality of electrodes.

8. The method of claim 7 wherein the plurality of electrodes further comprises:
at least one energized electrode, the energized electrode engaged with a voltage or current source; and
at least one floating electrode, the floating electrode contributing electrical energy in an absence of electrical communication with the voltage or current source.

9. The method of claim 8 wherein the floating electrodes exhibit a constant and unknown voltage with a zero net current.

10. The method of claim 1 wherein computing the strength further comprises applying a fast multi-pole method (FMM) computation with lower/upper (LU) factorization.

11. The method of claim 10 wherein computing the strength further comprises computing the strength based on 4 or 8 electrodes.

12. The method of claim 1 wherein computing the strength of the electrical energy is based on determining a surface charge density at a position of a neural axon in the treatment region.

13. The method of claim 1 wherein computing the strength of the electrical energy is based on a faceted volumetric representation of the treatment region.

14. The method of claim 1 further comprising computing the strength of the electrical energy based on a derivative of a value of the electric field.

15. A DBS (Deep Brain Stimulation) modeling and electrostimulation therapy device, comprising:
an interface to a scan medium for receiving a scan image of a treatment region;
a stimulation probe operable for insertion to a purported location within the scan image;
a modeling application configured to:
determine a position of a target region within the scan image relative to the purported location;
compute a strength of the electrical energy at the position based on the electric field at the position; and
conclude an efficacy resulting from activation of an electrode delivering the electrical energy resulting from the stimulation probe at the purported location; and
a DBS stimulator configured to receive voltage instructions based on activation of the electrode at a predetermined efficacy and energize the probe according to the voltage instructions following insertion into the treatment region.

16. The device of claim 15 wherein the modeling application is further configured to concluding the efficacy by comparing the computed strength to a threshold indicative of a therapeutic effect on the treatment region.

17. The device of claim 15 wherein the scan image is a pixelated structure including a plurality of pixels and the target region includes a pixel at the position.

18. The device of claim 1 wherein the stimulation probe includes a plurality of electrodes on the stimulation probe, and the modeling application computes the strength of the electrical energy contributed from each of the plurality of electrodes.

19. The device of claim 15 wherein the modeling application is further configured to compute the strength by applying a fast multi-pole method (FMM) computation with lower/upper (LU) factorization.

20. A computer program embodying program code on a non-transitory computer readable medium that, when executed by a processor, performs steps for implementing a method for directing an electrostimulation therapy, the method comprising:
receiving a scan image of a treatment region;
determining a purported location of a stimulation probe inserted within the scan image;
determining a position of a target region within the scan image relative to the purported location;
computing a strength of the electrical energy at the position based on the electric field at the position; and
concluding an efficacy resulting from activation of an electrode delivering the electrical energy resulting from the stimulation probe at the purported location.
